(19) United States
(12) Patent Application Publication
(10) Pub. No.: US 2004/0038300 A1
(43) Pub. Date: Feb. 26, 2004

(54) Method for producing and identifying appropriate effectors of target molecules using substance libraries

(76) Inventors: Stefan Kienle, Frankfurt (DE); Norbert Windhab, Hofheim (DE); Christoph Brucher, Eschborn (DE); Karsten Kuhn, Dortmund (DE)

Correspondence Address: O'MELVENY & MEYERS, 114 PACIFICA, SUITE 100, IRVINE, CA, 92618, US

(21) Appl. No.: 10/362,030

(22) PCT Filed: Aug. 7, 2001
(86) PCT No.: PCT/EP01/09103

(30) Foreign Application Priority Data
Aug. 17, 2000 (DE) 100 40 289.5

(51) Int. Cl. C12Q 1/68; G01N 33/53; G01N 33/543

(52) U.S. Cl. 435/7.1; 435/6; 436/518

(57) ABSTRACT

Process for preparing and determining suitable effectors of target molecules using substance libraries

The present invention relates to a process for phenomenonologically describing target substances using characterized substance libraries and to a process for selecting components of a combinatorial substance library for screening active compounds, and to the characterized and combinatorially produced substance libraries themselves.

The present invention relates to a process for phenomenonologically describing target substances using characterized substance libraries and to a process for selecting components of a combinatorial substance library for screening active compounds, and to the characterized and combinatorially produced substance libraries themselves.

In general, the search for active compounds which are therapeutically or diagnostically active begins with the identification and validation of suitable target substances. The investigation of such biological targets with regard to substances which are potentially pharmacologically active frequently turns out to be very difficult.

The identification of a suitable target is frequently followed, in accordance with known methods of the prior art, by the selective, where appropriate combinatorial, synthesis of individual compounds or compound libraries [L. Van Hijfte, G. Marciniak, N. Froloff, J. Chromatogr. B 1999, 725, 3-15], which are analyzed, in vitro or in vivo assays, for their biological activity against the target.

The synthetically produced test substances are selected such that their physicochemical properties, such as the lipophilicity parameter log P, the acid constant pKₐ or the solubility L, homogeneously cover a property range which is as large as possible [H. Matter, J. Med. Chem. 1997, 40, 1219-1229]. A relatively small group of potentially active substances is then selected by biological screening, frequently by means of simple binding assays, from this library of compounds, which is as diverse as possible and which is consequently either incomplete or extremely extensive.

Proceeding from the specific physicochemical properties of these potentially active substances, defined compound libraries of restricted diversity are then in turn synthesized and investigated for their biological activity against the target. This then results in one or more model structures which are validated in further in vitro and in vivo assays.

Finally, the active substances are examined for their ADMET properties (ADMET stands for ability to be absorbed (A), ability to be distributed (D), ability to be metabolized (M), ability to be excreted (E) and toxicity for organisms or cell formations) before the substances are passed over into the phase of clinical investigation.

The implementation of such a process, which is based on the try and error principle, is correspondingly elaborate and cost-intensive. In particular, it is necessary to carry out a large number of individual experiments in order to identify suitable binding partners for the target. It is only at a very late stage in the development phase that it is possible to carry out a further optimization of the biologically active substances, the success of which optimization is frequently of crucial importance for determining the usability of the substances as pharmacological active compounds, for example.

High-throughput screening offers the possibility of handling and evaluating a relatively large number of substances to be tested. In the biological high-throughput screening of combinatorial compound libraries on solid phase, which libraries have been prepared, for example, using the split-couple-recombine method, it is possible to identify active substances either by directly analyzing the immobilized substance or by coding the solid-phase beads during synthesis and reading the code of the active beads. However, the biological high-throughput screening of combinatorial compound libraries in solution requires a multistep deconvolution and an elaborate synthesis of sublibraries, which are always defined. Frequently, individual compounds are also prepared in a highly parallel and automated manner and conveyed to the high-throughput screening as individual compounds, in order to be able to directly identify active substances [D. L. Venton, C. P. Woodbury, Chemom. Intell. Lab. Syst. 1999, 48,131-150].

In this connection, potentially active components of the test compounds, which are subjected to further processing, and nonactive components often have to be distinguished arbitrarily [G. W. Caldwell, Curr. Opin. Drug Discovery Dev. 2000, 30-41]. Frequently, the most biologically active 30% of the components of a compound library, or components which are subjectively active compound-like as regards molecular backbone chain and the functional side chains, are selected [B. Ladd, Mod. Drug Discovery 2000,1, 46-52].

For these reasons, aside from using the try and error methods, attempts are made to develop methods for designing active compounds rationally. The methods which should in particular be mentioned here are molecular modeling and SAR analysis [A. Ajay, W. P. Walters, M. A. Murcko, J. Med. Chem. 1998, 41, 3314-3324; E. Hodgkin, K. Andrews-Cramer, Mod. Drug Discovery 2000, 3, 55-60]. A large number of computer programs can nowadays be obtained for this purpose [W. A. Warr, J. Chem. Inf. Comput. Sci. 1997, 37, 134-140]. However, thus far, it has only been possible to develop a few active compounds using the purely rational methods for specifically designing biologically active substances; in practice, these methods have not thus far proved to be a success. Furthermore, in order to design active compounds rationally, the target has to be very well characterized; it is frequently even necessary to know the three-dimensional structure of the target.

The present invention is based on the object of making available a selective process for developing biologically and/or chemically active substances without having to have recourse to rational molecule design.

A characterized library according to the invention, which library contains a large number of substances which potentially have binding activity, is used for this purpose. Within the meaning of this invention, substances which potentially have binding activity are understood as being molecules which are able to interact with other compounds, in particular with nucleic acids, proteins or peptides. These molecules include, for example, low molecular weight substances, such as carboxylic acids, amines, esters, aldehydes, ketones, acetals and heterocycles, such as alkaloids, and lipids, saccharides, steroids and other natural products; however, it is also possible to use peptides and proteins, such as antibodies or peptoids, and also their homodimers or heterodimers or homomultimers or heteromultimers, or known agonists and antagonists of proteins.

In addition, the library is supplemented with a selection of substances which potentially have binding activity and which as homogeneously as possible, over a broad property range, cover predetermined physicochemical properties, such as size, lipophilicity or polarity. A suitable selection can, for example, be made with the aid of (J. M. Blaney, E. J. Martin, Current Opinion in Chemical Biology 1997, 1, 54-59; H. Matter, J. Med. Chem. 1997, 40, 1219-1229).

The library composed of substances which potentially have binding activity is characterized using test substances and thereby characterized. Suitable test substances are all the known proteins, polypeptides or nucleic acids, the structure of at least one agonist or antagonist of which is known. Preferred test substances are nucleic acids, proteins and peptides which are already well characterized, such as receptors, antibodies, enzymes, transcription factors, ion channels or coding or gene-regulatory DNA sequences, such as promoters or operators. Particularly preferred test substances are any compounds which are known to the skilled person as being therapeutic targets.

The library is characterized by determining the pattern with which the test substances bind to the substances possessing binding activity in the library. The contact can be effected either homogeneously in solution or heterogeneously, with the test substance being immobilized on a solid phase.

In the homogeneous assay, the test substance and the library to be characterized are in each case dissolved in a suitable solvent and brought into interaction. It is advantageous for the experiment to be carried out under defined conditions, such as precisely defined concentrations of the test substance and of the substances which potentially have binding activity or, for example, the reproducible use of physiological solution conditions. Subsequently, samples are removed from the solution and the static binding pattern of the test substance is determined, by way of the decrease in the concentration of the substances which possess binding activity and which are derived from the library, e.g. by means of electrospray ionization mass spectrometry (ESI-MS), nanospray ESI-MS, matrix-assisted laser desorption ionization mass spectrometry (MALDI) or time-of-flight secondary ion mass spectrometry (TOF-SIMS). It is possible to separate the sample which has been removed chromatographically before massspectrometrically determining the binding patter; however, the sample can also be subjected to mass spectrometric analysis directly.

Preferably, the contact in homogeneous solution takes place in a dialysis unit. Initially, all the substances which potentially have binding activity in the library to be characterized are present in a defined starting concentration which is preferably far above the concentration of the test substance. Consequently, it is primarily only the substances in the library which have the highest binding activity which bind, whereas the less active components do not enter into any binding in this competitive situation. During the course of the dialysis, samples can be removed after defined time intervals and the intensities of the mass signals of the individual components of the library can be determined, in a time-resolved manner, using ESI-MS, nanospray ESI-MS, MALDI or TOF-SIMS. These samples can be analyzed either without any further purification or after purification, which is preferably chromatographic.

A component, which has binding activity, of the library to be characterized and the test substance can be separated either in the spectrometer or, preferably, chromatographically on line. If, as the dialysis progresses, the concentration of the library falls below a particular value, a noncompetitive situation, in which even less active substances can bind, then arises. This chronologically dynamic experimental procedure supplies additional information due to the concentration gradient of the substances which potentially have binding activity which is produced during the chronological course of the measurement.

In the nonhomogeneous assay, test substances are bound to a solid phase, such as magnetic polymer beads. The library to be characterized is added as different, defined concentrations to the immobilized test substance such that both noncompetitive and competitive systems arise. After the solid phase, together with the bound active components from the library, has been separated off, mass spectrometric methods are used to determine either the MSL components which have remained in the solution or the bound components, after they have been eluted from the solid phase.

The library can be characterized, i.e. characterized within the meaning of this invention, by using different test substances to repeatedly determine the static binding pattern, or the chronologically dynamic binding pattern, of the substances from the library which possess binding activity. The degree to which the binding behavior of the library is characterized is directly proportional to the number of test substances which are used for training the library.

The test substance is selected in a problem-orientated manner; thus, if this library is to be used for investigating proteinaceous target substances, preferred test substances are well characterized native or artificial proteins.

Pattern recognition, in particular cluster analysis, is employed as a mathematical tool for analyzing these data (K. Backhaus, B. Erichson, W. Plinke, R. Wieber, Multivariate Analysemethoden [Multivariat Analytical Methods], 9th edition, 2000, Springer Verlag; D. T. Stanton, T. W. Morris, S. Roychoudhury, C. N. Parker, J. Chem. Inf. Comput. Sci. 1999, 39, 21-27; P. C. Jurs, Science 1986, 232, 1219-1224). If a characterized library composed of n components which potentially have binding activity is characterized with m test substances, a defined point in an n-dimensional space, which is spanned by the n components, can then be assigned to each test substance.

In the ideal case, the test substances which are selected cover the entire, spanned n-dimensional space which represents the components of the characterized library as points.

If a target substance is exposed to a characterized library according to the invention, its binding pattern can be determined in analogy with the methods described for the test substances. In this connection, the binding pattern which is determined describes the target substance phenomenonologically. If, for example, lipophilic regions are present in the target substance, corresponding substances possessing binding activity from the characterized library then bind; if polar regions are accessible, polar substances from the library are then bound. On the basis of the binding pattern which is determined, a specific point in the n-dimensional space which is spanned by the individual components can then be assigned to the target substance.

Points which lie close together in the library-specific n-dimensional space are assigned to chemical target substances or test substances which exhibit similar binding behavior.

The invention furthermore relates to a process for phenomenonologically describing target substances using a characterized library according to the invention which is composed of substances which potentially have binding activity, in which process the target compound to be investigated is brought into interaction with the characterized library and the binding pattern, which is produced by the specific interactions, of the target substance is subsequently determined. On the basis of the binding pattern which is determined, a point in the n-dimensional space of the characterized library, which point depicts the binding behavior of the target substance, is assigned to the target substance using the above-described method. The test substances which are represented by adjacent points in the n-dimensional space of the characterized library, and which because of this exhibit a similar binding behavior, can now be determined. It is then possible, from the known agonists or antagonists of the test substances, to identify physicochemical descriptors which promote specific binding with the target substance being investigated.

A great advantage of the present invention is that a newly identified target structure, for which neither agonists, antagonists or other binding partners has to be known, can be correlated with a group of test substances which possess structurally similar features and/or physicochemically similar properties. This means, conversely, that it is possible, preceding from the structural features and physicochemical properties of the known agonists, antagonists and non-binders of the similar test substances, to draw conclusions with regard to the physicochemical and structural requirements of potential agonists and antagonists of the target structure being investigated. On the basis of the information which has been obtained, it is possible to select compounds which can be investigated for having a specific interaction with the target substance to be investigated. Consequently, in contrast to the try-and-error principle, the process according to the invention makes it possible to select physicochemical descriptors which can be employed in the search for substances (effectors) which are active with regard to an unknown target substance without having to have recourse to rational molecule design.

Interesting target substances are, in particular, proteins which can be assigned to the appearance of specific diseases; in this connection, it is of no significance whether the three-dimensional structure is known or whether their biochemical behavior has been elucidated.

Other target structures of interest are DNA sequences which are active in gene regulation, such as promoters or operators.

The descriptors which have been identified using the above-described process can be used, for example, for preparing a library, which is generated combinatorially-synthetically (J. M. Blaney, E. J. Martin, Current Opinion in Chemical Biology 1997, 1, 54-59) and which is composed of potential effectors. When generating such a library, it is possible to take into account other descriptors which possess known physicochemical properties, for example structural elements which promote the ability of active compounds to pass through membranes or structural elements which improve the biological or physiological ability to be degraded or excreted (P. J. Sinko, Current Opinion in Drug Discovery & Development 1999, 2, 42-48).

Within the meaning of this invention, an effector is a biologically or chemically active substance which interacts specifically with the target substance to be investigated and influences its function. Examples of effectors are inhibitors, activators or inducers of enzymes, or coenzymes, transcription factors or repressors.

Consequently, the invention additionally relates to the use of descriptors, which have been determined using a characterized library according to the invention, for preparing a library composed of potential effectors and to the selectively generated libraries for identifying suitable effectors of the target

Suitable effectors can now be identified using methods which are known to a skilled person.

1. A process for phenomenonologically describing target molecules, comprising the following procedural steps:
a) contacting a target substance to be investigated with a characterized substance library composed of substances which potentially have binding activity,
b) determining the pattern with which the target substance to be investigated binds to the substances possessing binding activity,
c) identifying known test substances whose binding behavior is as similar as possible to that of the target substance to be investigated and determining the known agonists and/or antagonists of these test substances.

2. The process as claimed in one of the preceding claims, characterized in that the library was characterized using proteins, peptides or nucleic acids as test substances.

3. The process as claimed in one of the preceding claims, characterized in that the binding pattern is determined mass spectrometrically.

4. The process as claimed in one of the preceding claims, characterized in that the binding behaviors of the target substance and the test substance are compared by means of patent recognition and mathematical evaluation in accordance with cluster analysis.

5. A substance library containing substances which potentially have binding activity, characterized in that the library is characterized using test compounds.

6. A substance library containing substances which potentially have binding activity, characterized in that the library contains substances which bind known therapeutic targets.

7. A substance library as claimed in claim 5 or 6, characterized in that the substances which potentially have binding activity are carboxylic acids, amines, esters, aldehydes, ketones, acetals and heterocycles, such as alkaloids, and lipids, saccharides, steroids and other natural products, and also peptides and proteins, such as antibodies or peptoids and also their homodimers or heterodimers or homomultimers or heteromultimers or their agonists and antagonists.

8. A substance library as claimed in one of claims 5 to 7, characterized in that the substances which potentially have binding activity cover predetermined physicochemical properties over a broad property range.

9. The use of a characterized substance library as claimed in claims 5 to 8 for preselecting descriptors as the basis for constructing libraries composed of potential effectors.

10. The use of the process as claimed in claims 1 to 4 for preselecting descriptors as the basis for constructing libraries composed of potential effectors.

11. A substance library, which can be obtained by combinatorial synthesis from descriptors which have been determined using a process as claimed in one of claims 1 to 4.

12. A substance library as claimed in claim 11, characterized in that the compounds which are generated by means of combinatorial synthesis are varied using descriptors.

13. A process for finding suitable effectors for a target substance to be investigated, comprising the following procedural steps:
a) generating a library as claimed in claims 10 to 12 which is composed of potential effectors which contain structural features of agonists and/or antagonists which have been determined in accordance with a process as claimed in one of claims 1 to 4,
b) contacting the target substances to be investigated with this library, and
c) identifying binding partners of the target substance to be investigated.

14. The process as claimed in claim 10, characterized in that the binding partners are subsequently tested for their effector properties.
